(19) United States
(12) Patent Application Publication
(10) Pub. No.: US 2019/0208064 A1
(43) Pub. Date: Jul. 4, 2019

(54) ELECTRONIC DEVICE AND METHOD FOR IMAGE PROCESSING BASED ON CONTEXT INFORMATION

(71) Applicant: Samsung Electronics Co., Ltd., Suwon-si (KR)

(72) Inventors: Nari IM, Yongin-si (KR); Jaegon KIM, Hwaseong-si (KR); Yongju LEE, Suwon-si (KR); Mincheol HWANG, Seoul (KR); Jiyoon PARK, Yongin-si (KR)

(21) Appl. No.: 16/219,052

(22) Filed: Dec. 13, 2018

(30) Foreign Application Priority Data
Jan. 2, 2018 (KR) 10-2018-0000411

(51) Int. Cl. H04N 1/00; H04N 1/32; G06T 5/00; G06K 9/62

(52) U.S. Cl. CPC H04N 1/00244; H04N 1/0097; G06K 9/6201; G06T 5/001; G06K 9/6217; H04N 1/32518

(57) ABSTRACT

An electronic device for image processing is provided. The electronic device includes a communication circuit, a memory, and a processor configured to receive a request related to transmission of a first image from a first external device to a second external device, and based on the request, identify a policy of the second external device associated with a processing of the first image. When the policy satisfies a first specified condition, a second image is transmitted to the second external device, wherein at least one object included in a raw image corresponding to the first image is changed in accordance with a first technique in the second image. When the policy satisfies a second specified condition, a third image is transmitted to the second external device, wherein at least one object included in the raw image is changed in accordance with a second technique in the third image.

### CROSS-REFERENCE TO RELATED APPLICATION(S)

This application is based on and claims priority under 35 U.S.C. § 119 of a Korean patent application number 10-2018-0000411, filed on Jan. 2, 2018, in the Korean Intellectual Property Office, the disclosure of which is incorporated by reference herein in its entirety.

### BACKGROUND

### 1. Field

The disclosure relates to an electronic device and method for acquiring an image through a camera and processing the acquired image based on context information such as policy or attribute information of an external device to which the image is to be transmitted.

### 2. Description of Related Art

An electronic device capable of processing an image acquires a raw image through an image sensor and process the acquired raw image through an image signal processor (ISP). The ISP can process the raw image by using an image quality improvement algorithm, thus providing an image with improved quality. Specifically, the ISP can perform various kinds of processing such as white balance adjustment, color adjustment (e.g., color matrix, color correction, color enhancement), color filter array interpolation, noise reduction processing or sharpening, and image enhancement (e.g., high-dynamic-range (HDR), face detection). An image outputted from the ISP may have, for example, a YUV format. In addition, an image outputted from the ISP may be compressed, for example, using JPEG technique, and then stored in the electronic device.

Meanwhile, an image processing cloud system is being used for image backup and new media content creation. In this system, an image may be uploaded to a cloud server, and a computer vision-based technique such as image matching that is difficult to perform in a client device may be applied to the uploaded image. Using machine learning based software, for example, the cloud server can perform image recognition.

The above information is presented as background information only to assist with an understanding of the disclosure. No determination has been made, and no assertion is made, as to whether any of the above might be applicable as prior art with regard to the disclosure.

### SUMMARY

Aspects of the disclosure are to address at least the above-mentioned problems and/or disadvantages and to provide at least the advantages described below. Accordingly, an aspect of the disclosure is to provide an electronic device and method for acquiring an image through a camera and processing the acquired image based on context information such as policy or attribute information of an external device to which the image is to be transmitted.

Another aspect of the disclosure is to provide an electronic device that may identify context information associated with acquiring an image and process the acquired image in accordance with the identified contextual information. Specifically, in order to back up, edit, develop, or generate an image acquired from an image sensor, the electronic device may acquire or determine image correction information (e.g., information about a method of processing an image) by using context information corresponding to the image. The context information may include, for example, information about an external device which is a destination to which an image is to be transmitted, and policy or attribute information about image processing required by the external device. For example, the electronic device may process an image in consideration of a case where the external device has a policy of requiring a specific image processing scheme or prohibiting arbitrary image processing.

Additional aspects will be set forth in part in the description which follows and, in part, will be apparent from the description, or may be learned by practice of the presented embodiments.

In accordance with an aspect of the disclosure, an electronic device is provided. The electronic device may include a communication circuit, a memory, and at least one processor functionally connected to the communication circuit and the memory. The at least one processor may be configured to control the communication circuit to receive a request related to transmission of a first image from a first external device to a second external device, based on the request, identify a policy of the second external device associated with a processing of the first image, and when the policy satisfies a first specified condition, control the communication circuit to transmit a second image to the second external device, wherein at least one object included in a raw image corresponding to the first image is modified in accordance with a first technique, wherein the second image comprises the at least one object being modified by the first technique, wherein, when the policy satisfies a second specified condition, control the communication circuit to transmit a third image to the second external device, and wherein at least one object included in the raw image corresponding to the first image is modified in accordance with a second technique, and the third image comprises the at least one object being modified by the second technique.

In accordance with another aspect of the disclosure, an electronic device is provided. The electronic device includes a communication circuit, a memory, and at least one processor functionally connected to the communication circuit and the memory. The at least one processor may be configured to control the communication circuit to receive a request related to transmission of a first image from a first external device to a second external device, based on the request, identify a raw image corresponding to the first image and attribute information associated with the second external device, and when the attribute information satisfies a first specified condition, control the communication circuit to transmit a second image to the second external device, wherein at least one object included in the raw image is changed in accordance with a specific technique, wherein in the second image comprises the at least one object being modified by the specific technique, wherein, when the attribute information satisfies a second specified condition, control the communication circuit to transmit the first image to the second external device, and wherein at least one object included in the raw image is not changed in accordance with the specific technique in the first image.

In accordance with another aspect of the disclosure, an electronic device is provided. The electronic device includes a memory, a camera, a communication circuit, a display, and at least one processor electrically connected to the memory, the camera, the communication circuit, and the display. The memory may include instructions that cause, when executed, the at least one processor to acquire a first image, to set an external electronic device to which the first image is to be transmitted, identify policy or attribute information about image processing required by the external electronic device, correct the first image to a second image, based on the identified policy or attribute information, and control the communication circuit to transmit the second image to the external electronic device.

In accordance with another aspect of the disclosure, the electronic device for processing an image, based on context information corresponding to the image is provided. For example, the context information may include information about a key entered into the electronic device before and/or after the image acquisition, information about a menu or application executed before and/or after the image acquisition, information about a motion detected by the electronic device before and/or after the image acquisition, information about objects contained in the image, information about a previously applied image-processing technique in connection with an object contained in the acquired image, and image format information. Through this, the electronic device may select an object requiring image processing and also process the selected object in accordance with the user's intention.

Other aspects, advantages, and salient features of the disclosure will become apparent to those skilled in the art from the following detailed description, which, taken in conjunction with the annexed drawings, discloses various embodiments of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a block diagram illustrating an electronic device in a network environment according to an embodiment of the disclosure;

FIG. 2 is a block diagram illustrating a camera module according to an embodiment of the disclosure;

FIG. 3 is a conceptual diagram illustrating operations of an electronic device and an external electronic device according to an embodiment of the disclosure;

FIG. 4 is a block diagram illustrating an electronic device and an external electronic device according to an embodiment of the disclosure;

FIG. 5 is a block diagram illustrating an electronic device and an external electronic device according to an embodiment of the disclosure;

FIG. 6 is a flow diagram illustrating a method for processing an image based on context information of an electronic device according to an embodiment of the disclosure;

FIG. 7 is a flow diagram illustrating a method for selecting and assuring an image processing target and technique based on context information of an electronic device according to an embodiment of the disclosure;

FIG. 8 is a flow diagram illustrating a method for processing an image based on policy or attribute information of an external electronic device at an electronic device (e.g., a cloud server) according to an embodiment of the disclosure;

FIG. 9 is a flow diagram illustrating a method for processing an image based on first context information (e.g., an accident scene) of an electronic device according to an embodiment of the disclosure;

FIG. 10 is a flow diagram illustrating a method for processing an image based on second context information (e.g., telemedicine) of an electronic device according to an embodiment of the disclosure; and

FIG. 11 is a flow diagram illustrating a method for processing an image based on first context information (e.g., travel) of an electronic device according to an embodiment of the disclosure.

Throughout the drawings, like reference numerals will be understood to refer to like parts, components, and structures.

### DETAILED DESCRIPTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

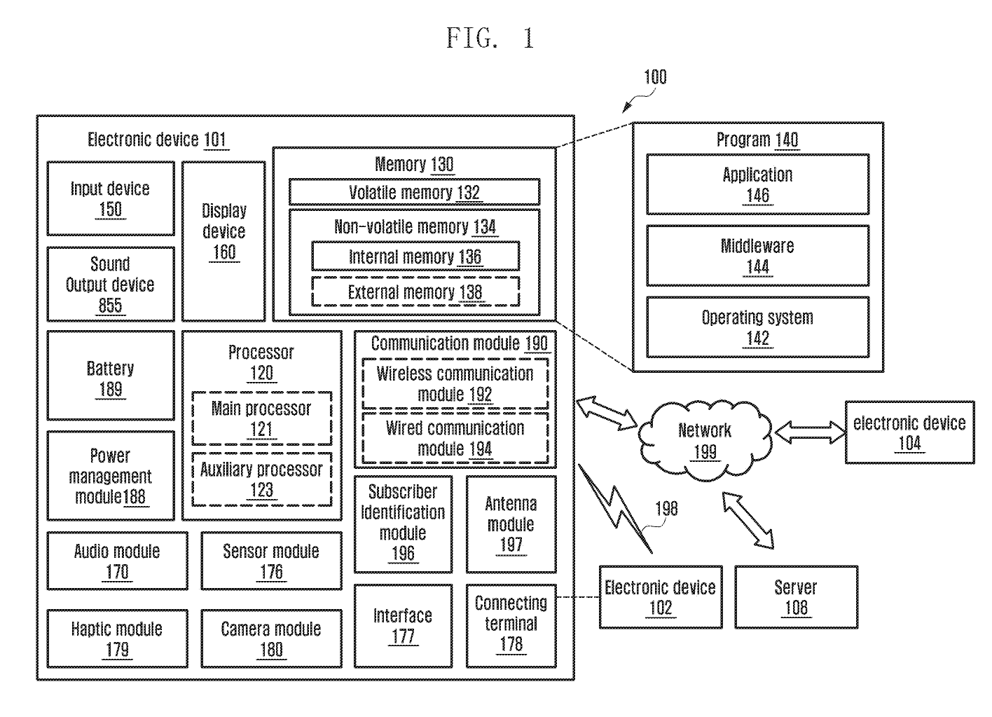

FIG. 1 is a block diagram illustrating an electronic device in a network environment according to an embodiment of the disclosure. FIG. 1 illustrates a block diagram 100.

Referring to FIG. 1, the electronic device 101 in the network environment 100 may communicate with an electronic device 102 via a first network 198 (e.g., a short-range wireless communication network), or an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input device 150, a sound output device 855, a display device 160, an audio module 170, a sensor module 176, an interface 177, a haptic module 179, a camera module 180, a power management module 188, a battery 189, a communication module 190, a subscriber identification module (SIM) 196, or an antenna module 197. In some embodiments, at least one (e.g., the display device 160 or the camera module 180) of the components may be omitted from the electronic device 101, or one or more other components may be added in the electronic device 101. In some embodiments, some of the components may be implemented as single integrated circuitry. For example, the sensor module 176 (e.g., a fingerprint sensor, an iris sensor, or an illuminance sensor) may be implemented as embedded in the display device 160 (e.g., a display).

The processor 120 may execute, for example, software (e.g., a program 140) to control at least one other component (e.g., a hardware or software component) of the electronic device 101 coupled with the processor 120, and may perform various data processing or computation. According to one embodiment, as at least part of the data processing or computation, the processor 120 may load a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), and an auxiliary processor 123 (e.g., a graphics processing unit (GPU), an image signal processor (ISP), a sensor hub processor, or a communication processor (CP)) that is operable independently from, or in conjunction with, the main processor 121. Additionally or alternatively, the auxiliary processor 123 may be adapted to consume less power than the main processor 121, or to be specific to a specified function. The auxiliary processor 123 may be implemented as separate from, or as part of the main processor 121.

The auxiliary processor 123 may control at least some of functions or states related to at least one component (e.g., the display device 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123.

The memory 130 may store various data used by at least one component (e.g., the processor 120 or the sensor module 176) of the electronic device 101. The various data may include, for example, software (e.g., the program 140) and input data or output data for a command related thererto. The memory 130 may include the volatile memory 132 or the non-volatile memory 134.

The program 140 may be stored in the memory 130 as software, and may include, for example, an operating system (OS) 142, middleware 144, or an application 146. The input device 150 may receive a command or data to be used by other component(s) (e.g., the processor 120) of the electronic device 101, from the outside (e.g., a user) of the electronic device 101. The input device 150 may include, for example, a microphone, a mouse, a keyboard, or a digital pen (e.g., a stylus pen).

The sound output device 855 may output sound signals to the outside of the electronic device 101. The sound output device 855 may include, for example, a speaker or a receiver. The speaker may be used for general purposes, such as playing multimedia or playing record, and the receiver may be used for incoming calls. According to an embodiment, the receiver may be implemented as separate from, or as part of the speaker.

The display device 160 may visually provide information to the outside (e.g., a user) of the electronic device 101. The display device 160 may include, for example, a display, a hologram device, or a projector and control circuitry to control a corresponding one of the display, hologram device, and projector. According to an embodiment, the display device 160 may include touch circuitry adapted to detect a touch, or sensor circuitry (e.g., a pressure sensor) adapted to measure the intensity of force incurred by the touch.

The audio module 170 may convert a sound into an electrical signal and vice versa. According to an embodiment, the audio module 170 may obtain the sound via the input device 150, or output the sound via the sound output device 855 or a headphone of an external electronic device (e.g., an electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). According to an embodiment, the connecting terminal 178 may include, for example, a HDMI connector, a USB connector, a SD card connector, or an audio connector (e.g., a headphone connector).

The haptic module 179 may convert an electrical signal into a mechanical stimulus (e.g., a vibration or a movement) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to one embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device via the first network 198 (e.g., a short-range communication network, such as Bluetooth™, wireless-fidelity (Wi-Fi) direct, or infrared data association (IrDA)) or the second network 199 (e.g., a long-range communication network, such as a cellular network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 192 may identify and authenticate the electronic device 101 in a communication network, such as the first network 198 or the second network 199, using subscriber information (e.g., international mobile subscriber identity (IMSI)) stored in the subscriber identification module 196.

The antenna module 197 may transmit or receive a signal or power to or from the outside (e.g., the external electronic device) of the electronic device 101. According to an embodiment, the antenna module 197 may include an antenna including a radiating element composed of a conductive material or a conductive pattern formed in or on a substrate (e.g., a printed circuit board (PCB)). According to an embodiment, the antenna module 197 may include a plurality of antennas. In such a case, at least one antenna appropriate for a communication scheme used in the communication network, such as the first network 198 or the second network 199, may be selected, for example, by the communication module 190 (e.g., the wireless communication module 192) from the plurality of antennas. The signal or the power may then be transmitted or received between the communication module 190 and the external electronic device via the selected at least one antenna. According to an embodiment, another component (e.g., a radio frequency integrated circuit (RFIC)) other than the radiating element may be additionally formed as part of the antenna module 197.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. Each of the electronic devices 102 and 104 may be a device of a same type as, or a different type, from the electronic device 101. According to an embodiment, all or some of operations to be executed at the electronic device 101 may be executed at one or more of the external electronic devices 102, 104, or 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, or client-server computing technology may be used, for example.

The electronic device according to various embodiments may be one of various types of electronic devices. The electronic devices may include, for example, a portable communication device (e.g., a smartphone), a computer device, a portable multimedia device, a portable medical device, a camera, a wearable device, or a home appliance. According to an embodiment of the disclosure, the electronic devices are not limited to those described above.

It should be appreciated that various embodiments of the disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. It is to be understood that a singular form of a noun corresponding to an item may include one or more of the things, unless the relevant context clearly indicates otherwise. As used herein, each of such phrases as “A or B,” “at least one of A and B,” “at least one of A or B,” “A, B, or C,” “at least one of A, B, and C,” and “at least one of A, B, or C,” may include any one of, or all possible combinations of the items enumerated together in a corresponding one of the phrases. As used herein, such terms as “1st” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with,” “coupled to,” “connected with,” or “connected to” another element (e.g., a second element), it means that the element may be coupled with the other element directly (e.g., wiredly), wirelessly, or via a third element.

As used herein, the term “module” may include a unit implemented in hardware, software, or firmware, and may interchangeably be used with other terms, for example, “logic,” “logic block,” “part,” or “circuitry”. A module may be a single integral component, or a minimum unit or part thereof, adapted to perform one or more functions. For example, according to an embodiment, the module may be implemented in a form of an application-specific integrated circuit (ASIC).

Various embodiments as set forth herein may be implemented as software (e.g., the program 140) including one or more instructions that are stored in a storage medium (e.g., internal memory 136 or external memory 138) that is readable by a machine (e.g., the electronic device 101). For example, a processor (e.g., the processor 120) of the machine (e.g., the electronic device 101) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The machine-readable storage medium may be provided in the form of a non-transitory storage medium. Wherein, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to an embodiment, a method according to various embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various embodiments, each component (e.g., a module or a program) of the above-described components may include a single entity or multiple entities. According to various embodiments, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

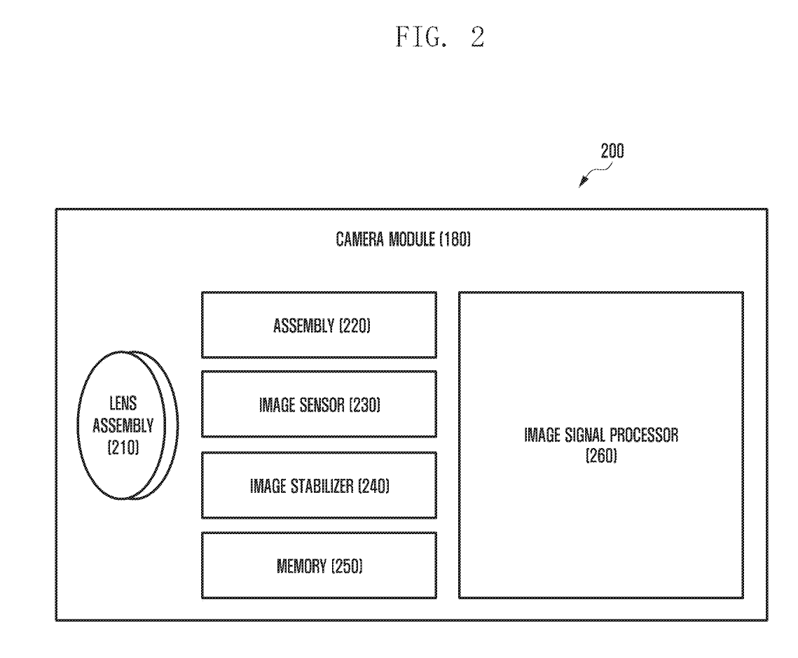

FIG. 2 is a block diagram illustrating a camera module according to an embodiment of the present disclosure.

Referring to FIG. 2, an embodiment of the disclosure includes an electronic device 200 including a cameral module 180. The camera module 180 may include a lens assembly 210, an assembly 220, an image sensor 230, an image stabilizer 240, a memory 250, and an image signal processor 260 that are interconnected.

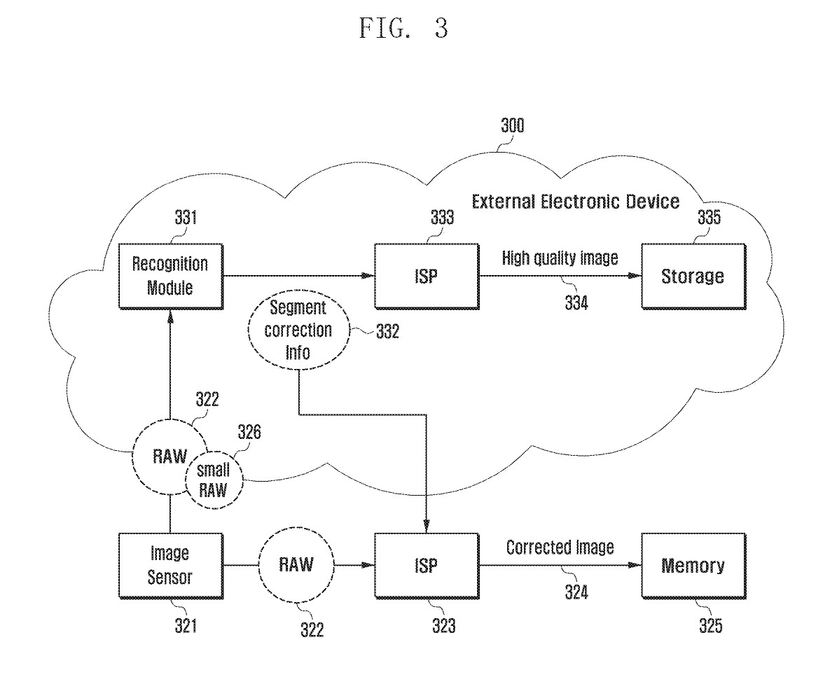

FIG. 3 is a conceptual diagram illustrating operations of an electronic device and an external electronic device according to an embodiment of the disclosure.

Referring to FIG. 3, the electronic device 101 may include an image sensor 321, an image signal processor (ISP) 323, and a memory 325. The external electronic device 300 may include a recognition module 331, an ISP 333, and a storage 335. The recognition module 331 may be a logical module and may be implemented as a processor of the external electronic device 300. Also, the ISP 333 may be implemented as a processor of the external electronic device 300. For example, the processor of the external electronic device 300 may perform both recognition and image processing. Although not shown, the electronic device 101 may include a communication module (e.g., the communication module 190 of FIG. 1, a communication circuit, or a transceiver) capable of transmitting and receiving data to and from the external electronic device 300. Also, the external electronic device 300 may include a communication module capable of transmitting and receiving data to and from the electronic device 101.

In various embodiments, the image sensor 321 (e.g., the image sensor 230 of FIG. 2) may acquire an image for an external object and generate a corresponding raw image 322. The image sensor 321 may deliver the raw image 322 to the ISP 323 (e.g., the ISP 260 of FIG. 2). In various embodiments, the image sensor 321 may generate a small raw image 326 and transmit the small raw image 326 to the external electronic device 300 through the communication module. In various embodiments, a processor (e.g., the processor 120) of the electronic device 101, instead of the image sensor 321, may generate the small raw image 326 and transmit the generated small raw image 326 to the external electronic device 300 via the communication module. The image sensor 321 may compress the raw image 322 for partial processing and store the compressed raw image 322 in an internal memory thereof. The recognition module 331 of the external electronic device 300 may acquire the small raw image 326 through the communication module and perform segmentation for dividing the small raw image 326 into at least one image segment.

In various embodiments, the recognition module 331 may recognize each of the at least one image segment generated as a result of segmentation. Also, the recognition module 331 may generate segment correction information 332 including information associated with the image segments, e.g., coordinate information or recognition result of the image segment. Then, the recognition module 331 may transmit the segment correction information 332 to the electronic device 101. The ISP 323 of the electronic device 101 may correct the raw image 322 by using the segment correction information 332, and thus a corrected image 324 may be generated. The corrected image 324 may have, for example, a YUV format. Also, the corrected image 324 may be stored in the memory 325. The electronic device 101 may store the corrected image 324 in the memory 325. Alternatively, the electronic device 101 may compress the corrected image 324, for example, in accordance with the JPEG technique, and store the compressed image in the memory 325.

In various embodiments, the raw image 322 provided from the image sensor 321 may be transmitted to the external electronic device 300 separately from the small raw image 326. Since the raw image 322 is greater in volume than the small raw image 326, the small raw image 326 may be first transmitted to the external electronic device 300, and the raw image 322 may be then transmitted to the external electronic device 300. For example, the raw image 322 may be transmitted to the external electronic device 300 while the ISP 323 performs the correction on the raw image 322. The raw image 322 may be uploaded to the external electronic device 300 in a state of being generated by the image sensor 321 or may be uploaded as a preprocessed image in which lens distortion compensation or noise removal has been performed. The external electronic device 300 may perform de-mosaic processing, image format modification, or preprocessing to increase an image recognition rate. The ISP 333 of the external electronic device 300 may correct the received raw image 322 by using the previously generated segment correction information 332 or using extended segment correction information. The raw image 322 may have a higher resolution than that of the small raw image 326, so that the ISP 333 of the external electronic device 300 may acquire more detailed extended segment correction information from a high quality image. The ISP 333 may generate the extended segment correction information by using both the previously generated segment correction information and the raw image 322. The ISP 333 may acquire a high quality image 334 by correcting the raw image 322 through the extended segment correction information. The high quality image 334 may be stored in the storage 335 of the external electronic device 300 and downloaded to the electronic device 101.

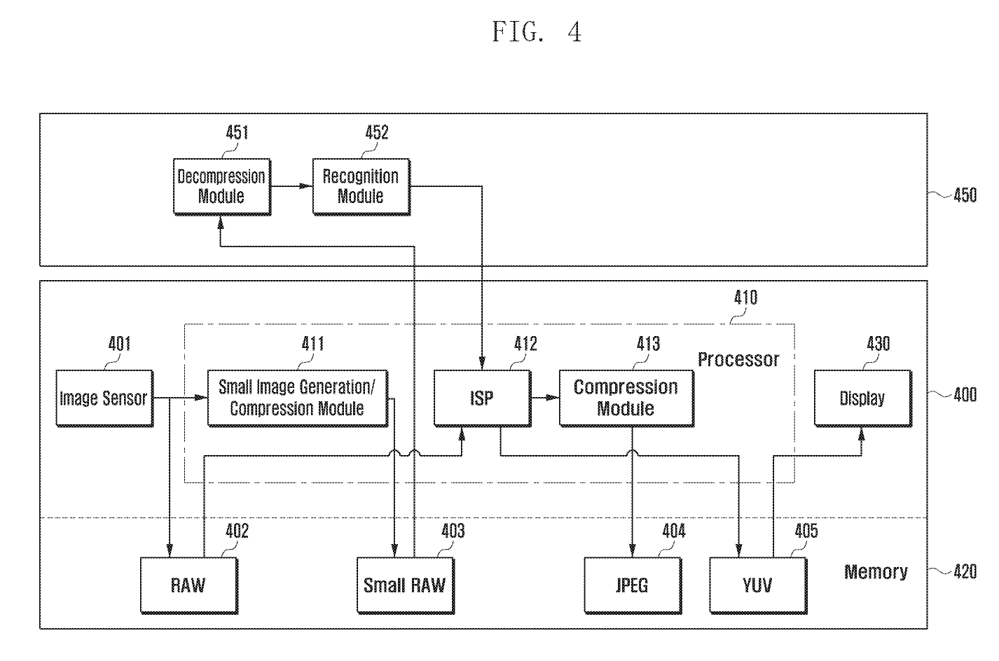

FIG. 4 is a block diagram illustrating an electronic device and an external electronic device according to an embodiment of the disclosure.

Referring to FIG. 4, the electronic device 400 (e.g., the electronic device 101 of FIG. 1 or 200 of FIG. 2) may include an image sensor 401, a processor 410, a memory 420, and a display 430. The external electronic device 450 may include a decompression module 451 and a recognition module 452. The operations performed by the decompression module 451 and the recognition module 452 may be performed by a processor (not shown) of the external electronic device 450.

In various embodiments, the image sensor 401 (e.g., the camera module 180 of FIG. 2) may capture an image of an external object and thereby generate a raw image 402. The raw image 402 may be temporarily or non-temporarily stored in the memory 420 (e.g., a dynamic random access memory (DRAM)). The processor 410 may include a small image generation and compression module 411, an ISP 412, and a compression module 413. The small image generation and compression module 411, the ISP 412, and the compression module 413 may be, for example, logic modules, and thus the operations thereof may be performed by the processor 410 (e.g., the processor 120 of FIG. 1). In a certain embodiment, at least one of the small image generation and compression module 411, the ISP 412, or the compression module 413 may be implemented as hardware in the processor 410. The small image generation and compression module 411 may receive the generated raw image 402 from the image sensor 401 and generate a small raw image 403 from the raw image 402. The small image generation and compression module 411 may compress the small raw image 403 and store the compressed small raw image 403 in the memory 420. The small raw image 403 may be temporarily or non-temporarily stored in the memory 420. A communication module (not shown) (e.g., the communication module 190 of FIG. 1) of the electronic device 400 may transmit the small raw image 403 stored in the memory 420 to the external electronic device 450.

In various embodiments, the decompression module 451 of the external electronic device 450 may receive the small raw image 403. As described above, the small raw image 403 may be in a compressed state, and the decompression module 451 may decompress the small raw image 403 and deliver the decompressed small raw image to the recognition module 452. Then, the recognition module 452 may recognize the decompressed small raw image. For example, the recognition module 452 may perform segmentation of the small raw image and thereby divide it into at least one image segment. In addition, the recognition module 452 may perform recognition of the image segment(s), based on one of various recognition algorithms, and thereby obtain a recognition result. The recognition module 452 may classify scenes of images and analyze reliability of image classification information and recognition information. The at least one image segment in the small raw image and the recognition result may be delivered as segment correction information to the electronic device 400. In various embodiments, the segment correction information may be composed to include at least one of image segment related information (having location (or coordinate) information and/or recognition result of the image segment), image classification information, texture information, or reliability information. This will be described later in more detail.

In various embodiments, the ISP 412 may generate a corrected image 405 by using the raw image 402 and the segment correction information. The corrected image 405 may have, for example, a YUV format. However, as understood by those skilled in the art, the format of the corrected image 405 is not limited. The corrected image 405 may be stored, for example, in a VRAM and also displayed on the display 430. The ISP 412 may deliver the corrected image 405 to the compression module 413. The compression module 413 may compress the received corrected image 405 and store a compressed image 404 in the memory 420. The compression module 413 may perform compression based on the JPEG technique, for example. However, as understood by those skilled in the art, there is no limitation in the compression technique.

In various embodiments, the external electronic device 450 may further include a processing circuit such as an ISP (not shown). In this case, using the segment correction information, the external electronic device 450 may generate pixel adjustment information for at least one pixel of the small raw image 403. For example, the external electronic device 450 may generate the pixel adjustment information by an effect (e.g., a tuning policy) corresponding to the segment correction information. The external electronic device 450 may deliver the pixel adjustment information to the electronic device 400. The ISP 412 may generate the corrected image 405 by applying the pixel adjustment information to the raw image 402. In this case, the ISP 412 may generate the corrected image 405 by simply adjusting at least one of brightness, contrast, color, or color temperature of at least one pixel in the raw image 402, based on the pixel adjustment information.

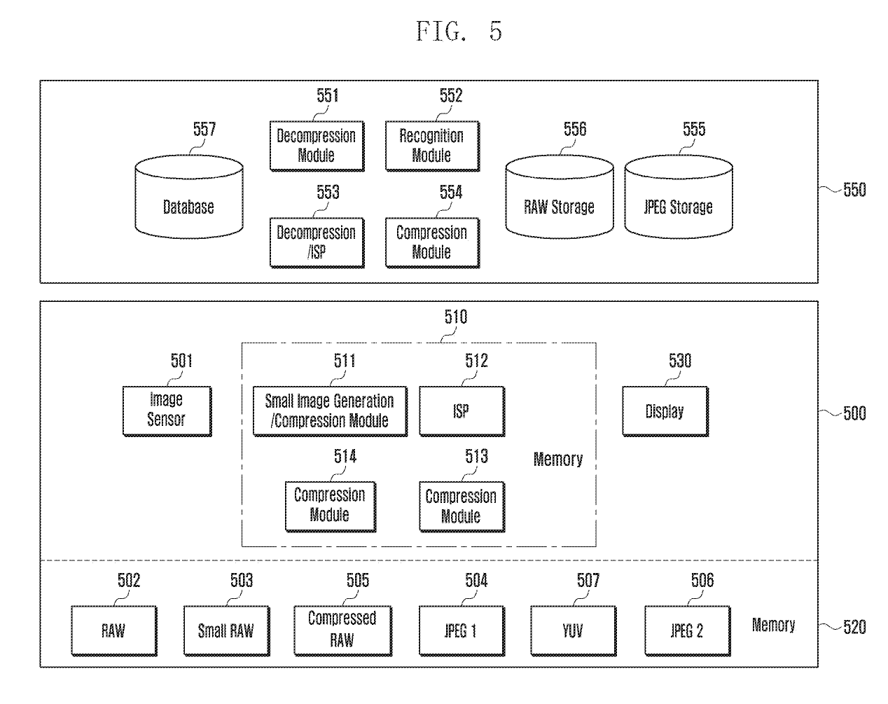

FIG. 5 is a block diagram illustrating an electronic device and an external electronic device according to an embodiment of the disclosure.

Referring to FIG. 5, the electronic device 500 (e.g., the electronic device 101) may include an image sensor 501, a processor 510, a memory 520, and a display 530. The processor 510 may include a small image generation and compression module 511, an ISP 512, a first compression module 513, and a second compression module 514. The external electronic device 550 may include a decompression module 551, a recognition module 552, a decompression and ISP 553, a compression module 554, a compressed image storage 555, a raw image storage 556, and a database 557.

The image sensor 501 may generate a raw image 502 and store the raw image 502 in the memory 520. The small image generation and compression module 511 may generate a small image, i.e., a small raw image 503, by using the raw image 502 and compress the small raw image 503. The compressed small raw image 503 may be stored in the memory 520. A communication module (not shown) of the electronic device 500 may transmit the small raw image 503 to a communication module (not shown) of the external electronic device 550. The decompression module 551 may receive the compressed small raw image 503 and decompress the received small raw image 503. The recognition module 552 may generate segment correction information by using the decompressed small raw image. The segment correction information may be transmitted to the ISP 512 of the electronic device 500 via the communication modules, and then the ISP 512 may generate a corrected image 507 by using the raw image 502 and the segment correction information. The corrected image 507 may have, for example, a YUV format. The corrected image 507 may be stored in the memory 520, for example, in the VRAM and also displayed on the display 530. The ISP 512 may deliver the corrected image 507 to the first compression module 513, which may compress the corrected image to generate a compressed image 504 and store the compressed image 504 in the memory 520.

In various embodiments, the second compression module 514 may compress the raw image 502 and store a compressed raw image 505 in the memory 520. Also, the compressed raw image 505 may be stored in the raw image storage 556 of the external electronic device 550 via the communication modules.

In various embodiments, the electronic device 101 may transmit the raw image 502 to the external electronic device 400 without compressing it. This non-compressed raw image may be stored in the database 557. In addition, the database 557 may further store a feature vector, which may be used in a correction process of the decompression and ISP 553 of the external electronic device 550. For example, the decompression and ISP 553 may receive the feature vector from the database 557, synthesize the feature vector with the raw image decompressed using segmentation information, and thereby generate a super-high-quality image. In addition, the decompression and ISP 553 may perform image quality enhancement by applying a predefined tuning policy (e.g., an effect) corresponding to image classification information, based on to the segmentation information. The feature vector may be a part of an object, such as a hair, in an image, or be an entire object such as a human face or a leaf. The feature vector may include a patch of a texture, a vector font, and the like. The decompression and ISP 553 may apply a predefined tuning policy corresponding to a category of the segment correction information in accordance with segmentation information of segment correction information generated based on the raw image. When failing to receive the feature vector, the decompression and ISP 553 may perform rendering of a feature vector on the basis of category information.

In various embodiments, the compressed raw image 505 may be decompressed and then stored in the database 557. The raw image contained in database 557 may be delivered to the recognition module 552 and the decompression and ISP 553. The recognition module 552 may perform recognition of the raw image. In addition, the recognition module 552 may generate extended segment correction information as a recognition result for the raw image. For example, the recognition module 552 may acquire the extended segment correction information from the raw image or alternatively generate the extended segment correction information by using previously generated segment correction information and the raw image together. The decompression and ISP 553 may decompress the compressed raw image and generate a corrected image by correcting the raw image.

In various embodiments, the decompression and ISP 553 may perform a correction process by using the recognition results received from the recognition module 552. The compression module 554 may compress the corrected image received from the decompression and ISP 553 in accordance with, for example, the JPEG compression technique. A compressed image 506 may be stored in the compressed image storage 555. Although the compressed image storage 555 is shown as “JPEG storage” in FIG. 5, this is merely denominated according to an employed compression technique and is not construed as a limitation. Also, the compressed image 506 may be stored in the memory 520 of the electronic device 500 via the communication modules. The decompression and the ISP 553 of the external electronic device 550 may have higher computing capability or higher resources than the ISP 512 of the electronic device 101 and thus may use a higher capacity correction algorithm. Therefore, the image 506 compressed from the corrected image by the external electronic device 550 may have a further enhanced image quality than that of the image 504 compressed from the corrected image 507 by the electronic device 500. The electronic device 500 may store both the compressed images 504 and 506 or replace the compressed image 504 stored therein with the compressed image 506.

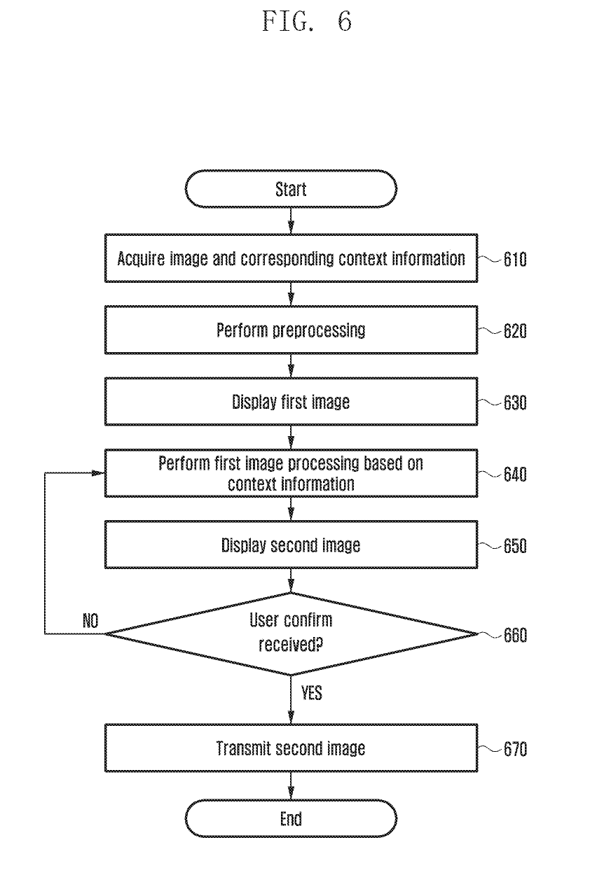

FIG. 6 is a flow diagram illustrating a method for processing an image based on context information of an electronic device according to an embodiment of the disclosure.

Referring to FIG. 6, at operation 610, the electronic device 101 may acquire an image and also acquire context information corresponding to the acquired image. For example, the electronic device 101 may acquire a captured image from the camera module 180 or acquire a stored image from the memory 130 or the server 108. For example, the context information corresponding to the image may include information about a key entered into the electronic device before and/or after the image acquisition, information about a menu or application executed before and/or after the image acquisition, information about a motion detected by the electronic device before and/or after the image acquisition, information about an object contained in the acquired image, information about a previously applied image-processing technique in connection with an object contained in the acquired image, and image format information. Thus, the context information may indicate all kinds of information other than pixel data in the image. For example, the context information may include information (e.g., black box information, car impact information, etc.) received from the external electronic device 102 or 104 connected to the electronic device 101, and may include information (images, user profiles, user preferences, etc.) stored in the server 108. Through this, the electronic device may select an object requiring image processing and also process the selected object in accordance with the acquired context information.

At operation 620, the electronic device 101 may perform preprocessing of the acquired image. That is, the electronic device 101 may process the acquired image to have a form suitable for data transmission or object recognition. For example, the electronic device 101 may compress the captured image and detect an object (e.g., a person, a thing, etc.) contained in the image. Of course, an object contained in the image may also be detected through the server 108 (e.g., a cloud server).

At operation 630, the electronic device 101 may display a first image. Specifically, the electronic device 101 may display the first image, which is pre-processed at operation 620, on the display 160. In various embodiments, the electronic device 101 may selectively display at least one of objects contained in the image, based on the acquired context information. For example, a certain object selected from the image at the preprocessing operation may be displayed differently from the other objects contained in the image or from the remaining image area.

At operation 640, the electronic device 101 may process the first image, based on the context information. For example, when a single image contains a plurality of objects, the electronic device 101 may perform image processing for all the objects by applying the same recipe (e.g., correction technique information or pixel adjustment information). In another example, when a single image contains a plurality of objects, the electronic device 101 may perform image processing for only a specific object with a specific recipe, based on the context information. That is, based on the context information, the electronic device 101 may determine a processing target (i.e., a correction target, a correction segment, a correction object, an adjustment target, an adjustment segment, an adjustment object, or the like) and a processing technique (i.e., a correction technique, scheme or method, an adjustment technique, scheme or method, recipe information, or the like). Then, the electronic device 101 may process the first image with the determined processing target and technique.

At operation 650, the electronic device 101 may display a second image. For example, the electronic device 101 may provide the second image as a result of processing the first image in accordance with the determined processing target and technique.

At operation 660, the electronic device 101 may receive a user's confirmation for the second image.

When failing to receive the user's confirmation for the second image at operation 660, the electronic device 101 may return to the operation 640 to process the first image again, based on the context information. For example, when the user is not satisfied with the second image, the electronic device 101 may not receive a response from the user for a given time. Alternatively, the electronic device 101 may receive a user's feedback that requests any other image processing method. Then, the context information acquired at operation 610 may be changed or updated to apply the user's feedback, and the electronic device 101 may process the first image again, based on the changed or updated context information.

When receiving the user's confirmation for the second image at operation 660, the electronic device 101 may transmit the second image at operation 670. For example, when the user approves the second image acquired based on the context information, the electronic device 101 may transmit the acquired second image to a certain destination (e.g., another electronic device 102 or 104 or the server 108). In this case, the server 108 may include, for example, an application server, an SNS server, a mail server, and/or the like.

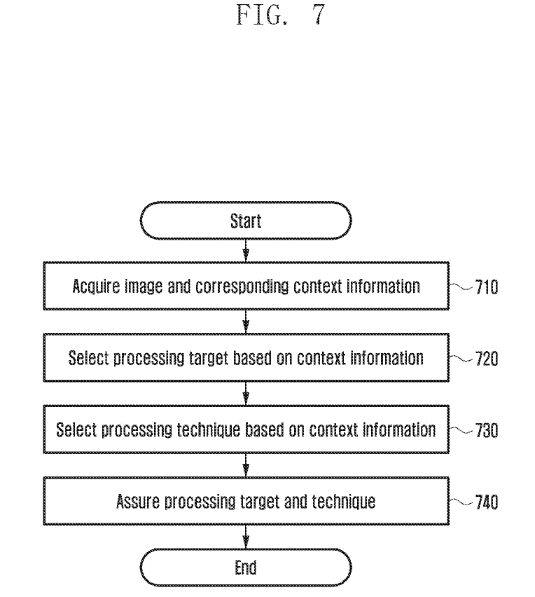

FIG. 7 is a flow diagram illustrating a method for selecting and assuring an image processing target and technique based on context information of an electronic device according to an embodiment of the disclosure.

Referring to FIG. 7, at operation 710, the electronic device 101 may acquire an image and also acquire context information corresponding to the acquired image. For example, the electronic device 101 may acquire a captured image from the camera module 180 or acquire a stored image from the memory 130 or the server 108. For example, the context information corresponding to the image may include information about a key entered into the electronic device before and/or after the image acquisition, information about a menu or application executed before and/or after the image acquisition, information about a motion detected by the electronic device before and/or after the image acquisition, information about an object contained in the acquired image, information about a previously image-processing technique in connection with an object contained in the acquired image, and image format information. Thus, the context information may indicate all kinds of information other than pixel data in the image. For example, the context information may include information (e.g., black box information, car impact information, etc.) received from the external electronic device 102 or 104 connected to the electronic device 101, and may include information (images, user profiles, user preferences, etc.) stored in the server 108. Through this, the electronic device may select an object requiring image processing and also process the selected object in accordance with the acquired context information.

At operation 720, the electronic device 101 may select a processing target, based on the context information. For example, the electronic device 101 may select, as the processing target, at least one of a plurality of objects contained in the image, based on the context information. In this example, the electronic device 101 may display the selected object(s) differently from the other objects contained in the image or from the remaining image area. Alternatively, the electronic device 101 may select the entire image as the processing target, based on the context information.

At operation 730, the electronic device 101 may select a processing technique, based on the context information. Specifically, the electronic device 101 may apply at least one processing technique to the at least one processing target selected at the operation 720. For example, when a single image contains a plurality of processing targets, the same processing technique may be applied to all the processing targets. Alternatively, based on the context information, a specific processing technique may be applied to only a specific processing target. For example, the image processing technique may include edge control, noise removal, specific color adjustment, overall color adjustment, hue adjustment, saturation adjustment, contrast adjustment, dynamic range expansion, size conversion of the entire or partial range, rotation, or any other technique. For each processing target, applied technique and intensity may be selectively adjusted.

At operation 740, the electronic device 101 may assure the selected processing target and the selected processing technique. For example, the electronic device 101 may insert a digital mark that warrants that a specific level corresponding to the context information (e.g., destination information, a purpose of using an image, an image format) is applied to the selected processing target and technique. For example, the digital mark may be stored in a form consistent with the intended use of the image according to the context information or stored in any other promised form. In addition, the digital mark may be included in a portion of the recipe or in a portion of the raw file. Accordingly, the electronic device 101 may acquire an image, determine a destination for which the image is to be used, and process the image to match the purpose and level of the image required at the destination. As discussed above in FIG. 6, the process may be repeatedly performed in accordance with a user's feedback on a process result.

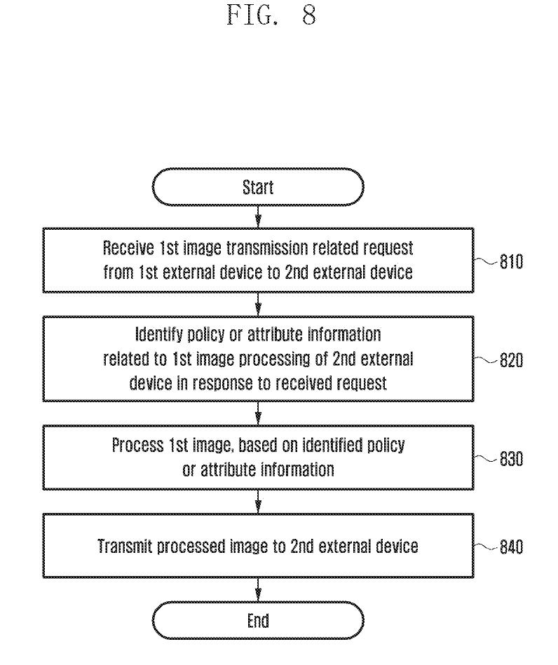

FIG. 8 is a flow diagram illustrating a method for processing an image based on policy or attribute information of an external electronic device at an electronic device (e.g., a cloud server) according to an embodiment of the disclosure.

Referring to FIG. 8, at operation 810, the electronic device (e.g., the server 108 of FIG. 1) may receive, from a first external device (e.g., the electronic device 101 of FIG. 1), a request related to transmission of a first image to a second external device (e.g., the electronic device 104 of FIG. 1). For example, a user of the first external device may transmit an image for a legal or medical purpose to the second external device via the electronic device.

At operation 820, in response to the received request, the electronic device (e.g., the server 108 of FIG. 1) may identify policy or attribute information associated with processing of the first image of the second external device (e.g., the electronic device 104 of FIG. 1). For example, the first image may be an image for a legal or medical use associated with the user of the first external device, and thus may be subject to the restriction of image processing. According to one embodiment, when the first image is an image for a legal use, processing the first image with an arbitrary technique may cause degradation in the admissibility of evidence of the first image. According to another embodiment, when the first image is an image for a medical use, processing the first image with an arbitrary technique may make it difficult to accurately diagnose a patient's state. Accordingly, the electronic device needs to process the first image after checking the policy or attribute information on the image processing of the second external device.

At operation 830, the electronic device (e.g., the server 108 in FIG. 1) may process the first image, based on the identified policy or attribute information. For example, the policy or attribute information required by the second external device may include an image format, a compression technique, the maintenance of an original image, and the like. In a certain embodiment, in addition to processing the first image, the electronic device may further perform the assurance that the processing on the first image is performed in accordance with the policy or attribute information required by the second external device. For example, the electronic device may insert a digital mark as an example of a warranty.

At operation 840, the electronic device (e.g., the server 108 of FIG. 1) may transmit the processed image to the second external device (e.g., the electronic device 104 of FIG. 1).

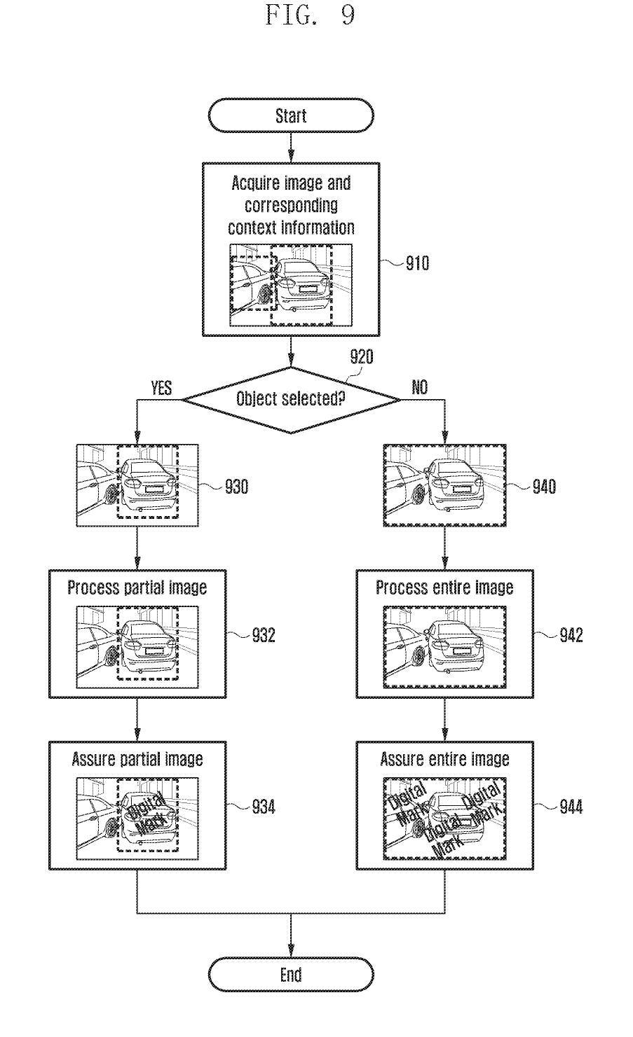

FIG. 9 is a flow diagram illustrating a method for processing an image based on first context information (e.g., an accident scene) of an electronic device according to an embodiment of the disclosure.

Referring to FIG. 9, at operation 910, the electronic device 101 may acquire an image and context information corresponding to the image. In addition, the electronic device 101 may detect objects contained in the acquired image through a preprocessing process. For example, as shown, the image indicates a traffic accident between two cars. In this example, the electronic device 101 may recognize two cars as different distinctive objects. Also, the electronic device 101 may acquire the context information corresponding to the image. For example, when the user executes an insurance company application, the electronic device 101 may obtain the context information that the user wants to capture an image of a traffic accident scene and submit it to an insurance company. Specifically, the electronic device 101 may recognize that the acquired image is for the purpose of insurance company submission, and thus may acquire policy or attribute information of the insurance company regarding an image processing technique, an image format, etc. adapted for the recognized purpose. In another example, the electronic device 101 may obtain information about the amount of impulse or movement with respect to the electronic device 101 before a certain time than capturing the image, and also recognize the contact of two cars through the captured image. Thus, in this example, the electronic device 101 may acquire the context information indicating a traffic accident.

At operation 920, the electronic device 101 may select an object as an image processing target, based on the context information corresponding to the image. For example, the electronic device 101 or a cloud server connected to the electronic device 101 may store information about a vehicle registered as the user's own. In this example, the electronic device 101 may obtain the vehicle information of the user as the context information corresponding to the image and use the context information in selecting the image processing target.

When a specific object is selected based on the context information at operation 920, the electronic device 101 may display the selected specific object to be distinguished from the other objects at operation 930. In this case, the selected specific object is considered as the image processing target.

When a specific object is not selected based on the context information at operation 920, the electronic device 101 may select the entire image as the image processing target at operation 940.

At operation 932, the electronic device 101 may perform partial image processing on the selected object. At this operation, when the image is, for example, a photo image to be provided as evidence of an accident to an insurance company, the electronic device 101 may select a processing technique that does not perform artificial processing on the image source.

At operation 942, the electronic device 101 may perform image processing on the entire image. At this operation, when the image is, for example, a photo image to be provided as evidence of an accident to an insurance company, the electronic device 101 may select a processing technique that does not perform artificial processing on the image source.

At operation 934, the electronic device 101 may assure the partially processed object or segment of the image. That is, the electronic device 101 may perform the assurance that a specific image processing level adapted for the context information (e.g., insurer submission image) is applied to the selected object and to the selected image processing technique. For example, the electronic device 101 may insert a digital mark as an example of a warranty. The digital mark may be stored in a form of a promised agreement with the insurer or be stored in a form of a flag. The digital mark may be included in part of information about a processing technique or in a portion of the raw image.

At operation 944, the electronic device 101 may assure the image-processed entire image. That is, the electronic device 101 may perform the assurance that a specific image processing level adapted for the context information (e.g., insurer submission image) is applied to the entire image and to the selected image processing technique. For example, the electronic device 101 may insert a digital mark as an example of a warranty. The digital mark may be stored in a form of a promised agreement with the insurer or be stored in a form of a flag. The digital mark may be included in part of information about a processing technique or in a portion of the raw image.

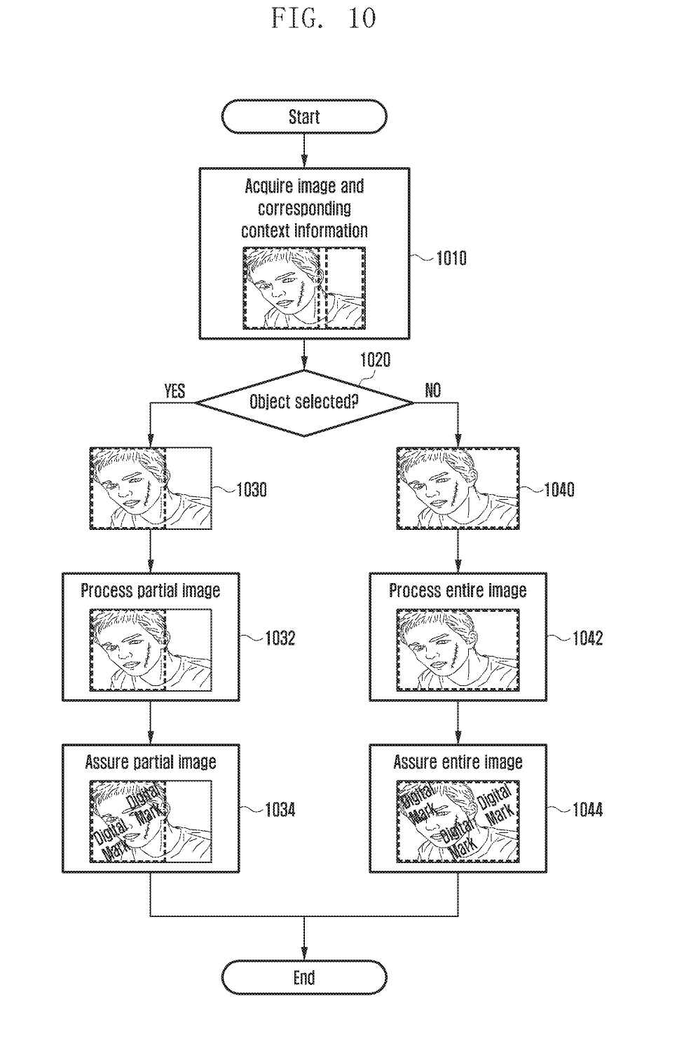

FIG. 10 is a flow diagram illustrating a method for processing an image based on second context information (e.g., telemedicine) of an electronic device according to an embodiment of the disclosure.

Referring to FIG. 10, at operation 1010, the electronic device 101 may acquire an image and context information corresponding to the image. In addition, the electronic device 101 may detect objects contained in the acquired image through a preprocessing process. For example, as shown, the image indicates an injured face. In this example, the electronic device 101 may recognize the injured face and clothes as different distinctive objects. Also, the electronic device 101 may acquire the context information corresponding to the image. For example, when the user executes a telemedicine application, the electronic device 101 may obtain the context information that the user wants to use a remote medical service for the injured face. Specifically, the electronic device 101 may recognize that the acquired image is for the purpose of telemedicine, and thus may acquire policy or attribute information of a medical service provider regarding an image processing technique, an image format, etc. adapted for the recognized purpose. In another example, the electronic device 101 may obtain a keyword “skin” or “skin care” entered or selected by the user, and may also recognize the context information that the user desires to receive medical care concerning the skin.

At operation 1020, the electronic device 101 may select an object as an image processing target, based on the context information corresponding to the image. For example, when the telemedicine is for a dermatologist or when the user inputs a face to a medical treatment site, the electronic device 101 may select a user's face as the object.

When a specific object is selected based on the context information at operation 1020, the electronic device 101 may display the selected specific object to be distinguished from the other objects at operation 1030. In this case, the selected specific object is considered as the image processing target.

When a specific object is not selected based on the context information at operation 1020, the electronic device 101 may select the entire image as the image processing target at operation 1040.

At operation 1032, the electronic device 101 may perform partial image processing on the selected object. At this operation, when the image is, for example, a medical image for telemedicine, the electronic device 101 may select a processing technique required in a remote medical service. For example, the electronic device 101 may process the partial image with a suitable technique such as lossless compression and 12-bit data processing.

At operation 1042, the electronic device 101 may perform image processing on the entire image. At this operation, when the image is, for example, a medical image for telemedicine, the electronic device 101 may select a processing technique required in a remote medical service. For example, the electronic device 101 may process the entire image with a suitable technique such as lossless compression and 12-bit data processing.

At operation 1034, the electronic device 101 may assure the partially processed object or segment of the image. That is, the electronic device 101 may perform the assurance that a specific image processing level adapted for the context information (e.g., telemedicine image) is applied to the selected object and to the selected image processing technique. For example, the electronic device 101 may insert a digital mark as an example of a warranty. The digital mark may be stored in a form of a promised agreement with the remote medical service or be stored in a form of a flag. The digital mark may be included in part of information about a processing technique or in a portion of the raw image.

At operation 1044, the electronic device 101 may assure the image-processed entire image. That is, the electronic device 101 may perform the assurance that a specific image processing level adapted for the context information (e.g., telemedicine image) is applied to the entire image and to the selected image processing technique. For example, the electronic device 101 may insert a digital mark as an example of a warranty. The digital mark may be stored in a form of a promised agreement with the remote medical service or be stored in a form of a flag. The digital mark may be included in part of information about a processing technique or in a portion of the raw image.

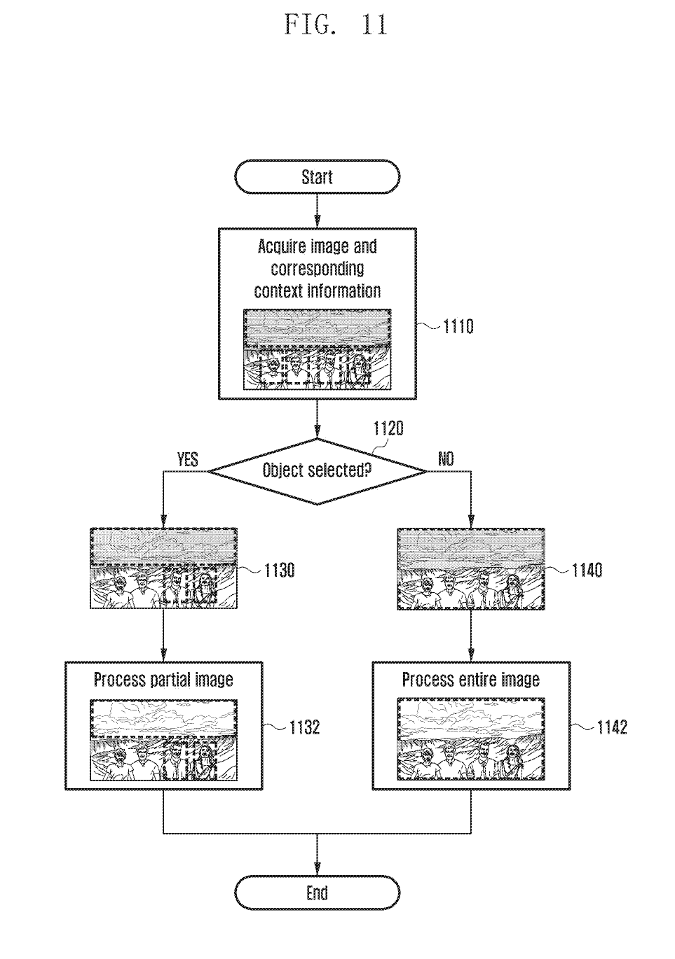

FIG. 11 is a flow diagram illustrating a method for processing an image based on first context information (e.g., travel) of an electronic device according to an embodiment of the disclosure.

Referring to FIG. 11, at operation 1110, the electronic device 101 may acquire an image and context information corresponding to the image. In addition, the electronic device 101 may detect objects contained in the acquired image through a preprocessing process. For example, as shown, the image indicates a family photograph. In this example, the electronic device 101 may recognize persons and a background as different distinctive objects. Also, the electronic device 101 may acquire the context information corresponding to the image. For example, the electronic device 101 may obtain, as the context information, weather information (e.g., cloudiness, fine dustiness, etc.) at the time when the image is photographed. In another example, the electronic device 101 may obtain a keyword “grandfather”, “sky”, or “me” entered or selected by the user, and thereby recognize the context information indicating which object the user wishes to process.

At operation 1120, the electronic device 101 may select an object as an image processing target, based on the context information corresponding to the image. For example, the electronic device 101 may select a specific object from among a plurality of objects contained in the image by utilizing object preference information, frequently photographed object information, etc. stored in a cloud server.

When a specific object is selected based on the context information at operation 1120, the electronic device 101 may display the selected specific object to be distinguished from the other objects at operation 1130. In this case, the selected specific object is considered as the image processing target.

When a specific object is not selected based on the context information at operation 1120, the electronic device 101 may select the entire image as the image processing target at operation 1140.

At operation 1132, the electronic device 101 may perform partial image processing on the selected object. For example, when the processing target is selected based on the user's keyword “grandfather”, “sky”, or “me”, the electronic device 101 may selectively process an object corresponding to the processing target. Also, the electronic device 101 may utilize, as the context information, information stored in the cloud server and related to how the user processes a portrait image. For example, based on the context information, the electronic device 101 may reapply a certain technique (e.g., correcting a white hair to a black hair and removing a wrinkle) that has been previously used for processing a grandfather image. In addition, cloudy sky with fine dust may be changed to blue sky.

At operation 1142, the electronic device 101 may perform image processing on the entire image. For example, the image of family picture may be calibrated to make it brighter, vivid, and vibrant.

Since the FIG. 11 embodiment relates to an image of everyday life which does not require any additional credibility, the electronic device 101 may omit the above-discussed operation of assuring the image.

According to various embodiments, an electronic device may comprise a communication module, a memory, and a processor functionally connected to the communication module and the memory. The processor may be configured to receive, using the communication module, a request related to transmission of a first image from a first external device to a second external device. In addition, based on the request, the processor may be configured to identify a policy of the second external device associated with processing of the first image. When the policy satisfies a first specified condition, the processor may be configured to transmit a second image to the second external device, wherein at least one object contained in a raw image corresponding to the first image is changed in accordance with a first technique in the second image. When the policy satisfies a second specified condition, the processor may be configured to transmit a third image to the second external device, wherein at least one object contained in the raw image is changed in accordance with a second technique in the third image.

The processor may be further configured to identify format information corresponding to the second external device, and to generate the second image in accordance with the identified format information.

The processor may be further configured to acquire destination information corresponding to the second external device, to identify a compression technique corresponding to the destination information, to compress the second image in accordance with to the identified compression technique, and to transmit the compressed second image to the second external device.

The processor may be further configured to generate processing result information according to processing of the first image, and to transmit the processing result information to the second external device in association with the second image.

The processor may be further configured to receive the raw image from the first external device, to transmit, to the first external device, the first or second technique for processing the received raw image, and to receive, from the first external device, the second image changed in accordance with the first technique or the third image changed in accordance with the second technique.

The processor may be further configured to request the second external device to provide selected object processing information to identify object processing information in accordance with a selected processing technique, and to receive, from the second external device, the selected object processing information.

According to various embodiments, an electronic device may comprise a communication module, a memory, and a processor functionally connected to the communication module and the memory. The processor may be configured to receive, using the communication module, a request related to transmission of a first image from a first external device to a second external device, to, based on the request, identify a raw image corresponding to the first image and attribute information associated with the second external device, to, when the attribute information satisfies a first specified condition, transmit a second image to the second external device, wherein at least one object contained in the raw image is changed in accordance with a specific technique in the second image, and to, when the attribute information satisfies a second specified condition, transmit the first image to the second external device, wherein at least one object contained in the raw image is not changed in accordance with the specific technique in the first image.

The processor may be further configured to identify format information corresponding to the second external device, and to generate the second image in accordance with the identified format information.

The processor may be further configured to acquire destination information corresponding to the second external device, to identify a compression technique corresponding to the destination information, to compress the second image in accordance with to the identified compression technique, and to transmit the compressed second image to the second external device.

The processor may be further configured to generate processing result information according to processing of the first image, and to transmit the processing result information to the second external device in association with the second image.

The processor may be further configured to receive the raw image from the first external device, to transmit, to the first external device, a specific technique corresponding to the first or second specified condition for processing the received raw image, and to receive, from the first external device, the second image changed in accordance with the specific technique or the first image not changed in accordance with the specific technique.

The processor may be further configured to request the second external device to provide selected object processing information to identify object processing information in accordance with a selected processing technique, and to receive, from the second external device, the selected object processing information.

According to various embodiments, an electronic device may comprise a memory, a camera module, a communication module, a display, and a processor electrically connected to the memory, the camera module, the communication module, and the display. The memory may include instructions that cause, when executed, the processor to acquire a first image, to set an external electronic device to which the first image is to be transmitted, to identify policy or attribute information about image processing required by the external electronic device, to correct the first image to a second image, based on the identified policy or attribute information, and to transmit the second image to the external electronic device.

The memory may include instructions that cause, when executed, the processor to receive a user feedback regarding the second image, and to transmit the second image to the external electronic device in response to the user feedback.

The memory may include instructions that cause, when executed, the processor to receive a user feedback regarding the second image, and to correct the first image to a third image in response to the user feedback.

The memory may include instructions that cause, when executed, the processor to insert assurance information about the second image.

The memory may include instructions that cause, when executed, the processor to determine, based on the policy or attribute information, whether at least one of objects contained in the first image is selectable, to, when the at least one object is selectable, acquire a correction technique for processing the selected at least one object, based on the policy or attribute information, and to generate the second image in which the selected at least one object is processed with the correction technique.

The memory may include instructions that cause, when executed, the processor to determine, based on the policy or attribute information, whether at least one of objects contained in the first image is selectable, to, when the at least one object is not selectable, acquire a correction technique for processing all the objects contained in the first image, based on the policy or attribute information, and to generate the second image in which all the objects contained in the first image are processed with the correction technique.

The memory may include instructions that cause, when executed, the processor to, when the policy or attribute information prohibits correction on the first image, transmit the first image to the external electronic device without correcting the first image to the second image.

The memory may include instructions that cause, when executed, the processor to acquire the first image via at least one of the camera module, the communication module, or the memory.

While the disclosure has been shown and described with reference to various embodiments thereof, it will be understood by those skilled in the art that various changes in form and details may be made therein without departing from the spirit and scope of the disclosure as defined by the appended claims and their equivalents.

What is claimed is:

1. An electronic device comprising:
a communication circuit;
a memory; and
at least one processor functionally connected to the communication circuit and the memory,
wherein the at least one processor is configured to:
control the communication circuit to receive a request related to transmission of a first image from a first external device to a second external device,
based on the request, identify a policy of the second external device associated with a processing of the first image, and
when the policy satisfies a first specified condition, control the communication circuit to transmit a second image to the second external device,
wherein at least one object included in a raw image corresponding to the first image is modified in accordance with a first technique,
wherein the second image comprises the at least one object being modified by the first technique,
wherein, when the policy satisfies a second specified condition, control the communication circuit to transmit a third image to the second external device, and
wherein at least one object included in the raw image corresponding to the first image is modified in accordance with a second technique, and the third image comprises the at least one object being modified by the second technique.

2. The electronic device of claim 1, wherein the at least one processor is further configured to:
identify format information corresponding to the second external device; and
generate the second image in accordance with the identified format information.

3. The electronic device of claim 1, wherein the at least one processor is further configured to:
acquire destination information corresponding to the second external device;
identify a compression technique corresponding to the destination information;
compress the second image in accordance with to the identified compression technique; and
control the communication circuit to transmit the compressed second image to the second external device.

4. The electronic device of claim 1, wherein the at least one processor is further configured to:
generate processing result information according to the processing of the first image; and
control the communication circuit to transmit the processing result information to the second external device in association with the second image.

5. The electronic device of claim 1, wherein the at least one processor is further configured to:
receive the raw image from the first external device;
control the communication circuit to transmit, to the first external device, the first or second technique for processing the received raw image; and
receive, from the first external device, the second image changed in accordance with the first technique or the third image changed in accordance with the second technique.

6. The electronic device of claim 1, wherein the at least one processor is further configured to:
request the second external device to provide selected object processing information to identify object processing information in accordance with a selected processing technique; and
receive, from the second external device, the selected object processing information.

7. An electronic device comprising:
a communication circuit;
a memory; and
at least one processor functionally connected to the communication circuit and the memory,
wherein the at least one processor is configured to:
control the communication circuit to receive a request related to transmission of a first image from a first external device to a second external device,
based on the request, identify a raw image corresponding to the first image and attribute information associated with the second external device, and
when the attribute information satisfies a first specified condition, control the communication circuit to transmit a second image to the second external device,
wherein at least one object included in the raw image is modified in accordance with a specific technique,
wherein the second image comprises the at least one object being modified by the specific technique,
wherein, when the attribute information satisfies a second specified condition, control the communication circuit to transmit the first image to the second external device, and
wherein at least one object included in the raw image is not changed in accordance with the specific technique.

8. The electronic device of claim 7, wherein the at least one processor is further configured to:
identify format information corresponding to the second external device; and
generate the second image in accordance with the identified format information.

9. The electronic device of claim 7, wherein the at least one processor is further configured to:
acquire destination information corresponding to the second external device;
identify a compression technique corresponding to the destination information;
compress the second image in accordance with to the identified compression technique; and
control the communication circuit to transmit the compressed second image to the second external device.

10. The electronic device of claim 7, wherein the at least one processor is further configured to:
generate processing result information according to the processing of the first image; and
control the communication circuit to transmit the processing result information to the second external device in association with the second image.

11. The electronic device of claim 7, wherein the at least one processor is further configured to:
receive the raw image from the first external device;
control the communication circuit to transmit, to the first external device, a specific technique corresponding to the first or second specified condition for processing the received raw image; and
receive, from the first external device, the second image changed in accordance with the specific technique or the first image not changed in accordance with the specific technique.

12. The electronic device of claim 7, wherein the at least one processor is further configured to:
request the second external device to provide selected object processing information to identify object processing information in accordance with a selected processing technique; and
receive, from the second external device, the selected object processing information.

13. An electronic device comprising:
a memory;
a camera;
a communication circuit;
a display; and
at least one processor electrically connected to the memory, the camera, the communication circuit, and the display,
wherein the memory includes instructions that cause, when executed, the at least one processor to:
acquire a first image,
set an external electronic device to which the first image is to be transmitted,
identify policy or attribute information about image processing required by the external electronic device,
correct the first image to a second image, based on the identified policy or attribute information, and
control the communication circuit to transmit the second image to the external electronic device.

14. The electronic device of claim 13, wherein the memory includes instructions that cause, when executed, the at least one processor to control the communication circuit to:
receive a user feedback regarding the second image; and
transmit the second image to the external electronic device in response to the user feedback.

15. The electronic device of claim 13, wherein the memory includes instructions that cause, when executed, the at least one processor to:
control the communication circuit to receive a user feedback regarding the second image; and
correct the first image to a third image in response to the user feedback.

16. The electronic device of claim 13, wherein the memory includes instructions that cause, when executed, the at least one processor to insert assurance information about the second image.

17. The electronic device of claim 13, wherein the memory includes instructions that cause, when executed, the at least one processor to:
determine, based on the policy or attribute information, whether at least one of objects contained in the first image is selectable;
when the at least one object is selectable, acquire a correction technique for processing the selected at least one object, based on the policy or attribute information; and
generate the second image in which the selected at least one object is processed with the correction technique.

18. The electronic device of claim 13, wherein the memory includes instructions that cause, when executed, the at least one processor to:
determine, based on the policy or attribute information, whether at least one of objects contained in the first image is selectable;
when the at least one object is not selectable, acquire a correction technique for processing all the objects contained in the first image, based on the policy or attribute information; and
generate the second image in which all the objects contained in the first image are processed with the correction technique.

19. The electronic device of claim 13, wherein the memory includes instructions that cause, when executed, the at least one processor to:
when the policy or attribute information prohibits correction on the first image, control the communication circuit to transmit the first image to the external electronic device without correcting the first image to the second image.

20. The electronic device of claim 13, wherein the memory includes instructions that cause, when executed, the at least one processor to:
acquire the first image via at least one of the camera, the communication circuit, or the memory.

21. The electronic device of claim 13, wherein the memory includes instructions that cause, when executed, the at least one processor to:
generate segment correction information including information associated with image segments.
